(19) United States
(12) Patent Application Publication
(10) Pub. No.: US 2026/0272689 A1
(43) Pub. Date: Sep. 17, 2026

(54) PARTIALLY CONNECTED SLEEVE FOR AN ENDOLUMINAL DEVICE HAVING AN UNCONNECTED PORTION FOR PORTAL ACCESS OF AN IMPLANTABLE DEVICE

(71) Applicant: W. L. Gore & Associates, Inc., Newark, DE (US)

(72) Inventors: Breanna Cornell, Flagstaff, AZ (US); Dustin C. Burkart, Bellemont, AZ (US)

(21) Appl. No.: 19/566,710

(22) Filed: Mar. 13, 2026

Related U.S. Application Data
(60) Provisional application No. 63/772,074, filed on Mar. 14, 2025.

(51) Int. Cl. A61F 2/97; A61M 25/00; A61M 25/06

(52) U.S. Cl. CPC A61F 2/97; A61F 2240/001; A61M 25/0009; A61M 25/0668

(57) ABSTRACT

An endoluminal device includes an implantable device and a constraining sleeve. The implantable device is transitionable between a collapsed, delivery configuration, an expanded, deployed configuration, and a partially deployed configuration between the collapsed, delivery configuration and the expanded, deployed configuration, and the implantable device has at least one opening providing access to an inner lumen of the implantable device. The constraining sleeve is removably disposed around and constraining the implantable device in the partially deployed configuration. The constraining sleeve has a first connected portion including a distal portion of the sleeve, a second connected portion including a proximal portion of the sleeve, and an unconnected portion located between the first and second connected portions providing access to the at least one opening.

### CROSS-REFERENCE TO RELATED APPLICATION

This application claims the benefit of Provisional Application No. 63/772,074, filed Mar. 14, 2025, which is incorporated herein by reference in its entirety for all purposes.

### FIELD

The present disclosure relates to endoluminal devices, and more specifically, to endoluminal devices with portal access.

### BACKGROUND

There is a need for advanced devices, tools, systems and methods used for the endoluminal treatment of vascular diseases in regions of branch vessels and main vessel junctions, including treatments requiring access to branch arteries.

### SUMMARY

Endoprosthesis devices and methods of manufacturing the same are disclosed herein. In one example (“Example 1”), an endoluminal device includes an implantable device and a constraining sleeve. The implantable device is transitionable between a collapsed, delivery configuration, an expanded, deployed configuration, and a partially deployed configuration between the collapsed, delivery configuration and the expanded, deployed configuration. The implantable device has at least one opening providing access to an inner lumen of the implantable device. The constraining sleeve is removably disposed around the implantable device. The constraining sleeve has a first connected portion including a distal portion of the constraining sleeve, a second connected portion including a proximal portion of the constraining sleeve, and an unconnected portion located between the first and second connected portions. The unconnected portion is adjacent to or overlaying the at least one opening when in the partially deployed configuration operable to provide access to the at least one opening from outside of the constraining sleeve.

In another example (“Example 2”) further to Example 1, the implantable device is self-expanding.

In another example (“Example 3”) further to Example 1 or 2, the constraining sleeve has two opposing edges, a first intermediate point located between the distal portion and the proximal portion, and a second intermediate point located between the distal portion and the proximal portion. The constraining sleeve has the first connected portion between the distal portion and the first intermediate point of the constraining sleeve, and the second connected portion between the proximal portion and the second intermediate point of the constraining sleeve.

In another example (“Example 4”) further to Example 3, the endoluminal device includes a single line releasably coupling together the two opposing edges of the constraining sleeve to constrain the implantable device in the partially deployed configuration.

In another example (“Example 5”) further to Example 4, the line forms the first connected portion of the constraining sleeve by connecting together the two opposing edges from the distal portion to the first intermediate point, the line forming the second connected portion by connecting together the two opposing edges from the second intermediate point to the proximal portion.

In another example (“Example 6”) further to Example 5, proximally retracting the line causes the implantable device to deploy from the distal portion toward the first intermediate point and to deploy from the second intermediate point toward the proximal portion.

In another example (“Example 7”) further to Example 4, the line connects together the two opposing edges from the first intermediate point to the distal portion defining the first connected portion of the constraining sleeve, and the line connects together the two opposing edges from the second intermediate point to the proximal portion defining the second connected portion.

In another example (“Example 8”) further to Example 7, proximally retracting the line causes the line to become unconnected or disengaged from the two opposing edges such that the implantable device deploys from the first intermediate point toward the distal portion and deploys from the second intermediate point toward the proximal portion so as to deploy the implantable device from the partially deployed configuration to the expanded, deployed configuration.

In another example (“Example 9”) further to Example 3, the endoluminal device includes a first line and a second line releasably coupling together the two opposing edges of the first and second connected portions, respectively, to constrain the implantable device in the partially deployed configuration. The first line connects together the two opposing edges from the first intermediate point to the distal portion defining the first connected portion of the constraining sleeve, and the second line connects together the two opposing edges from the second intermediate point to the proximal portion defining the second connected portion.

In another example (“Example 10”) further to Example 9, the first and second lines are separately retractable.

In another example (“Example 11”) further to Example 9 or 10, proximally retracting the first line causes the first line to become unconnected or disengaged from the two opposing edges such that the implantable device deploys from the first intermediate point toward the distal portion and proximally retracting the second line causes the second line to become unconnected or disengaged from the two opposing edges such that the implantable device deploys from the second intermediate point toward the proximal portion so as to deploy the implantable device from the partially deployed configuration to the expanded, deployed configuration.

In another example (“Example 12”) further to any one of Examples 1-11, the at least one opening includes at least a first opening, a second opening, and a third opening.

In one example (“Example 13”), a method of manufacturing an endoluminal device includes: providing an implantable device transitionable between a collapsed, delivery configuration, an expanded, deployed configuration, and a partially deployed configuration between the collapsed, delivery configuration and the expanded, deployed configuration, the implantable device having at least one opening providing access to an inner lumen of the implantable device; disposing a material around the implantable device; and partially connecting together opposing edges of the material to form a first connected portion including a distal portion of the constraining sleeve, a second connected portion including a proximal portion of the constraining sleeve, and an unconnected portion located between the first and second connected portions providing access to the at least one opening in the partially deployed configuration.

In another example (“Example 14”) further to Example 13, the partially connecting together opposing edges of the material includes: releasably coupling together the opposing edges of the constraining sleeve using a single line to constrain the implantable device in the partially deployed configuration.

In another example (“Example 15”) further to Example 14, the partially connecting together opposing edges of the material includes: forming, using the single line, the first connected portion of the constraining sleeve by connecting together the opposing edges from the distal portion to a first intermediate point; and forming, using the single line, the second connected portion by connecting together the opposing edges from a second intermediate point to the proximal portion. The first intermediate point is located between the distal portion and the proximal portion, and the second intermediate point is located between the distal portion and the proximal portion.

In another example (“Example 16”) further to Example 14, the partially connecting together opposing edges of the material includes: forming, using the single line, the first connected portion of the constraining sleeve by connecting together the opposing edges from a first intermediate point to the distal portion; and forming, using the single line, the second connected portion by connecting together the opposing edges from a second intermediate point to the proximal portion. The first intermediate point is located between the distal portion and the proximal portion, and the second intermediate point is located between the distal portion and the proximal portion.

In another example (“Example 17”) further to Example 13, the partially connecting together opposing edges of the material includes: releasably coupling together the opposing edges of the constraining sleeve using a first line and a second line to constrain the implantable device in the partially deployed configuration.

In another example (“Example 18”) further to Example 17, the partially connecting together opposing edges of the material includes: forming, using the first line, the first connected portion of the constraining sleeve by connecting together the opposing edges from a first intermediate point to the distal portion; and forming, using the second line, the second connected portion by connecting together the opposing edges from a second intermediate point to the proximal portion. The first intermediate point is located between the distal portion and the proximal portion, and the second intermediate point is located between the distal portion and the proximal portion.

In another example (“Example 19”) further to Example 18, the first and second lines are separately retractable.

In another example (“Example 20”) further to any one of Examples 13-19, the at least one opening includes at least a first opening, a second opening, and a third opening.

In one example (“Example 21”), a method of operating an endoluminal device includes: delivering the endoluminal device such that an implantable device is disposed in a collapsed, delivery configuration or a partially deployed configuration at a location within a vasculature, the implantable device having at least one opening providing access to an inner lumen of the implantable device, the implantable device having a constraining sleeve disposed around the implantable device, the constraining sleeve having a first connected portion including a distal portion of the constraining sleeve, a second connected portion including a proximal portion of the constraining sleeve, and an unconnected portion located between the first and second connected portions, the unconnected portion being adjacent to or overlaying the at least one opening when in the partially deployed configuration operable to provide access to the at least one opening from outside of the constraining sleeve; and at least partially removing the constraining sleeve from the implantable device to cause the implantable device to transition from the partially deployed configuration to an expanded, deployed configuration at the location within the vasculature.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a side view of an endoluminal device according to embodiments disclosed herein.

FIG. 2 is a schematic drawing of a sleeve for an endoluminal device according to embodiments disclosed herein.

FIG. 3 is a schematic drawing of a sleeve for an endoluminal device according to embodiments disclosed herein.

FIG. 4 is a schematic drawing of a sleeve for an endoluminal device according to embodiments disclosed herein.

FIG. 5 is a side view of a partially deployed implantable device according to embodiments disclosed herein.

FIG. 6 is a side view of a fully deployed implantable device according to embodiments disclosed herein.

FIG. 7 (prior art) is a top-down view of an endoluminal device implementing two or more sleeves as known in the art.

FIG. 8 is a flowchart of a process for manufacturing an endoluminal device according to embodiments disclosed herein.

### DETAILED DESCRIPTION

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

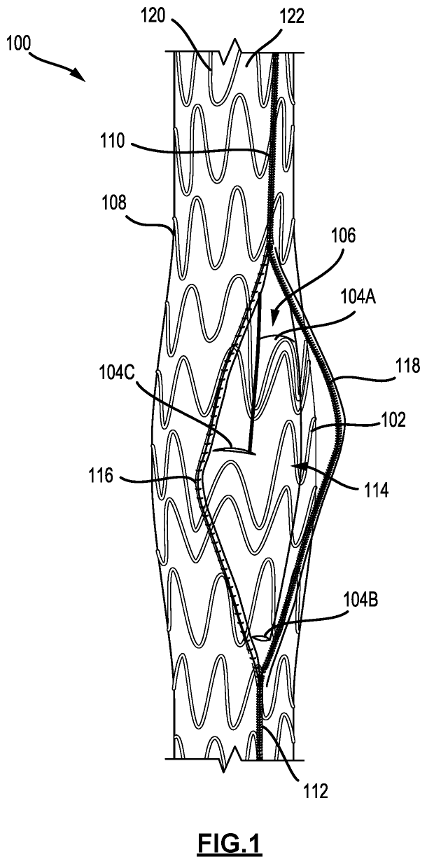

FIG. 1 shows an endoluminal device 100 according to embodiments disclosed herein. The device 100 includes an implantable device 102. The implantable device 102 is transitionable between a collapsed, delivery configuration and an expanded, deployed configuration having a greater diameter than the collapsed, delivery configuration. The implantable device 102 has at least one opening 104 providing access to an inner lumen 106 of the implantable device 102. The opening 104 may also be referred to as a portal or a fenestration in a sidewall of the implantable device 102.

In some examples, the at least one opening 104 includes at least a first opening 104A, a second opening 104B, and a third opening 104C. The third opening 104C is located between the first opening 104A and the second opening 104B along the longitudinal direction of the device 102. The opening 104A may be an opening that provides access of the lumen 106 of the implantable device 102 toward the direction of a brachiocephalic artery (BCA), hereinafter referred to as a BCA opening. The opening 104B may be an opening that provides access of the lumen 106 toward the direction of a left subclavian artery (LSA), hereinafter referred to as an LSA opening. The opening 104C may be an opening that provides access of the lumen 106 toward the direction of a left common carotid artery (LCCA), hereinafter referred to as an LCCA opening.

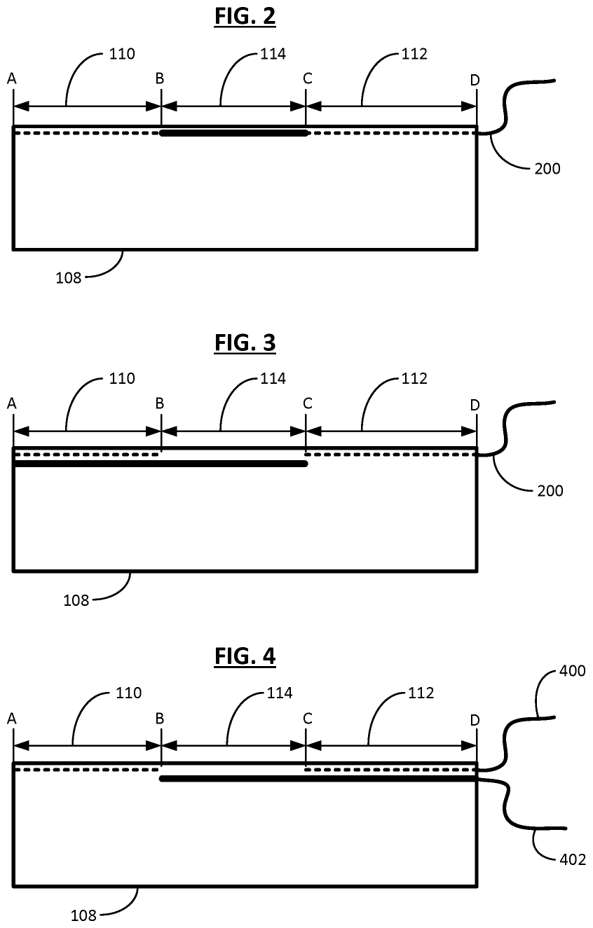

The endoluminal device 100 also includes a secondary constraining sleeve 108 that is removably disposed around and constraining the implantable device 102 in the collapsed, delivery configuration. A primary constraining sleeve (not shown) separate from the secondary constraining sleeve 108 is employed in ordered to restrain the implantable device 102 in a constrained state with a minimal diameter for delivery. When the primary constraining sleeve is removed, the secondary constraining sleeve 108 restrains the implantable device 102 in a partially deployed configuration. Referring to FIGS. 1 and 2, the secondary constraining sleeve 108 has a first connected portion 110 including a distal portion (point A) of the secondary constraining sleeve 108, a second connected portion 112 including a proximal portion(point B) of the sleeve 108, and an unconnected portion 114 (i.e., an open portion) located between the first and second connected portions 110, 112 providing access to the opening(s) 104. In some examples, the first connected portion 110 and the second connected portion 112 circumferentially surround the device 100, and the unconnected portion 114 includes at least one opening 104 such that it does not circumferentially surround the device 100 at the at least one opening 104 of the device 100. In some examples, the unconnected portion 114 is adjacent to or overlaying at least one opening 104 when the implantable device 102 is in the partially deployed configuration operable to provide access to at least one opening 104 from outside of the secondary constraining sleeve 108. In some examples, the implantable device 102 is self-expanding. In some examples, the implantable device 102 may expand with the assistance of an expanding device, such as a balloon. In some examples, the connected portions 110 and 112 are formed by coupling together two opposing edges 116 and 118 (or sides) of the sleeve 108 using one or more lines, which may be any suitable type of string, cord, thread, fiber, or wire which is capable of maintaining the sleeve in a substantially tubular shape. In some examples, the first connected portion 110 may be about 60 mm in length, the second connected portion 112 may be at least about 100 mm in length, and the unconnected portion 114 may be about 64 mm in length. In some examples, the connected portions are sewn, stitched, adhered, engaged, or otherwise physically coupled together, whereas the unconnected portions are unsewn, unstitched, unadhered, disengaged, or uncoupled from each other.

The implantable device 102 may be any suitable endoluminal devices such as a stent-graft with one or more stent components 120 and one or more graft members 122, such as with one or more graft members disposed over and/or under the stent components, which can dilate from a delivery configuration (collapsed, delivery configuration), through a range of larger intermediary configurations (intermediate deployment configuration), and toward a deployed configuration (expanded, deployed configuration) engaged with vessel walls at a treatment site. In some examples, the implantable device 102 is transitionable between the collapsed, delivery configuration and a partially deployed, intermediate deployment configuration having a greater diameter than the collapsed, delivery configuration, and between the intermediate deployment configuration and the expanded, deployed configuration. The intermediate deployment configuration is also referred to as a partially deployed configuration between the collapsed, delivery configuration and the expanded, deployed configuration. The expanded, deployed configuration has a greater diameter than the collapsed, delivery configuration and the intermediate deployment configuration, such that the intermediate deployment configuration has a smaller diameter than the expanded, deployed configuration.

Any suitable combination and configuration of stent component(s) and graft member(s) is within the scope of the present disclosure. For example, the stent-graft may be those used in GORE® TAG® Thoracic Branch Endoprosthesis (“TBE”), GORE® VIABAHN® Balloon Expandable (“VBX”) Endoprosthesis, Conformable GORE® TAG® Thoracic Endoprosthesis (“CTAG”), or GORER TAG® Conformable Thoracic Stent Graft with ACTIVE CONTROL System (“CMDS”), available from W. L. Gore & Associates, Inc. (Newark, DE). The sleeve may be made of any suitable an introducer sheath such as GORE® DrySeal Sheath, available from W. L. Gore & Associates, Inc.

Stent components can have various configurations such as, for example, rings, cut tubes, wound wires (or ribbons) or flat patterned sheets rolled into a tubular form. Stent components can be formed from metallic, polymeric or natural materials and can comprise conventional medical grade materials such as nylon, polyacrylamide, polycarbonate, polyethylene, polyformaldehyde, polymethylmethacrylate, polypropylene, polytetrafluoroethylene, polytrifluorochlorethylene, polyvinylchloride, polyurethane, elastomeric organosilicon polymers; metals such as stainless steels, cobalt-chromium alloys and nitinol and biologically derived materials such as bovine arteries/veins, pericardium and collagen. Stent components can also comprise bioresorbable materials such as poly(amino acids), poly(anhydrides), poly(caprolactones), poly(lactic/glycolic acid) polymers, poly(hydroxybutyrates) and poly(orthoesters). In some examples, the stent components may be GORE® Ascending Stent Graft (“ASG”), manufactured by W. L. Gore & Associates, Inc.

In addition, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system.

A biocompatible material for the graft components, discussed herein, may be used. In certain instances, the graft may include a fluoropolymer, such as a polytetrafluoroethylene (PTFE) polymer or an expanded polytetrafluoroethylene (ePTFE) polymer. In some instances, the graft may be formed of, such as, but not limited to, a polyester, a silicone, a urethane, an ultra high molecular weight polyethylene, a polyethylene terephthalate, fluoropolymers such as perfluoroelastomers and the like, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers. Other embodiments for a graft member material can include high strength polymer fibers such as ultra high molecular weight polyethylene fibers (e.g., Spectra®, Dyneema Purity®, etc.), aramid fibers (e.g., Technora®, etc.), and/or combinations thereof. The graft member may include a bioactive agent. In one embodiment, an ePTFE graft includes a carbon component along a blood contacting surface thereof. Any graft member which can be delivered by a catheter is in accordance with the present disclosure. Further detail of materials and general construction of stents, graft members and stent grafts are generally disclosed in U.S. Pat. Nos. 6,042,605; 6,361,637; and 6,520,986 all to Martin et al.

In FIGS. 2-4, the secondary constraining sleeve 108 is shown to have two opposing edges 116, 118, a first intermediate point (point B) located between the distal portion (point A) and the proximal portion (point D), and a second intermediate point (point C) located between the distal portion (point A) and the proximal portion (point D). The secondary constraining sleeve 108 has the first connected portion 110 located between the distal portion (A) and the first intermediate point (B) of the sleeve 108, and the second connected portion 112 is located between the proximal portion (D) and the second intermediate point (C) of the sleeve. In the figures, which shows the sleeve 108 from the side, the dotted lines represent the connected portions in which the line 200 is woven through both edges 116, 118 of the secondary constraining sleeve 108, whereas the bolded lines represent the stitched portions in which the line 200 is only woven through one of the two edges 116, 118 along the unused stitch holes at the edges.

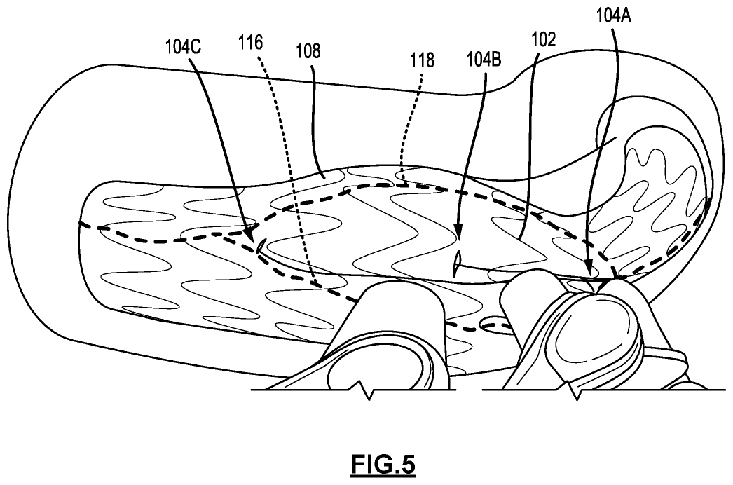

In FIGS. 2 and 3, the secondary constraining sleeve 108 has a single line 200 that releasably couples together the two opposing edges 116, 118 of the secondary constraining sleeve 108 in order to constrain the implantable device 102 (not shown) in the collapsed, delivery configuration. In FIG. 4, the secondary constraining sleeve 108 uses two lines 400 and 402 to releasably couple together the two opposing edges 116, 118 of the secondary constraining sleeve 108 in order to constrain the implantable device 102 in the collapsed, delivery configuration.

For example, in FIG. 2, the line 200 forms the first connected portion 110 of the sleeve by connecting together the two opposing edges 116, 118 from the distal portion (A) to the first intermediate point (B), and the line 200 forms the second connected portion 112 by connecting together the two opposing edges 116, 118 from the second intermediate point (C) to the proximal portion (D). As such, proximally retracting the line 200 causes the implantable device 102 to deploy from the distal portion (A) toward the first intermediate point (B) and to deploy from the second intermediate point (C) toward the proximal portion (D) so as to deploy the implantable device 102 from the partially deployed configuration to the expanded, deployed configuration.

The process of reducing the diameter of the implantable device 102 from the intermediate deployment configuration to the collapsed, delivery configuration, from the expanded, deployed configuration to the intermediate deployment configuration, or from the expanded, deployed configuration to the collapsed, delivery configuration such that the implantable device 102 is flush, is referred to as “crushing.” In some examples, implantable device 102 is crushed with the seamline of the secondary constraining sleeve 108 being centered to with respect to the opening(s) 104, leaving the middle, unconnected section open for access, also referred to as an unconnected portion or an open or unconnected portion 114, as the secondary constraining sleeve 108 opens in a distal-to-proximal direction.

In FIG. 3, the line 200 forms the first connected portion 110 of the secondary constraining sleeve 108 by connecting together the two opposing edges 116, 118 from the first intermediate point (B) to the distal portion (A). The line 200 then forms the second connected portion 112 by connecting together the two opposing edges 116, 118 from the second intermediate point (C) to the proximal portion (D). As such, proximally retracting the line 200 causes the implantable device 102 to deploy from the first intermediate point (B) toward the distal portion (A) and to deploy from the second intermediate point (C) toward the proximal portion (D).

In some examples, the second connected portion 112 of the secondary constraining sleeve 108 is connected first, from the second intermediate point (C) to the proximal portion (D). The deployment line 200 is then woven through the proximal single stitches and the midsection unused stitch holes. The secondary constraining sleeve 108 is then connected from the first intermediate point (B) to the distal portion (A) to form the first connected portion 110. This results in a distal-to-middle deployment followed by a proximal-to-middle deployment, with opposing deployment directions on the proximal and distal portions of the secondary constraining sleeve 108, such that both proximal and distal portions of the secondary constraining sleeve 108 deploy towards the midsection of the device. This configuration aids in preventing proximal and distal sleeve overhang. As the implantable device 102 deploys from the distal portion (A) and the proximal portion (D), the device 102 assumes a greater diameter than the midsection (B and D) and thereby pushes (or, alternatively, “shrugs” or “splits”) the sleeve 108 away from the proximal and distal portions (A and D).

In FIG. 4, a first line 400 and a second line 402 releasably couple together the two opposing edges 116, 118 of the secondary constraining sleeve 108 in order to constrain the implantable device 102 in the collapsed, delivery configuration. The first line 400 may couple together the opposing edges 116, 118 of the secondary constraining sleeve 108 at the first connected portion 110, and the second line 402 may couple together the opposing edges 116, 118 of the secondary constraining sleeve 108 at the second connected portion 112. For example, the first line 400 forms the first connected portion 110 of the sleeve 108 by connecting together the two opposing edges 116, 118 from the first intermediate point (B) to the distal portion (D). The second line 402 forms the second connected portion 112 by connecting together the two opposing edges 116, 118 from the second intermediate point (C) to the proximal portion (D). The first and second lines 400, 402 are separately retractable. Proximally retracting the first line 400 causes the implantable device 102 to deploy from the first intermediate point (B) toward the distal portion (A). Proximally retracting the second line 402 causes the implantable device 102 to deploy from the second intermediate point (C) toward the proximal portion (D). For example, proximally retracting the first line 400 may cause the first line to become unconnected or disengaged from the two opposing edges 116, 118 such that the implantable device 102 deploys from the first intermediate point (B) toward the distal portion (A) and proximally retracting the second line 402 may cause the second line to become unconnected or disengaged from the two opposing edges 116, 118 such that the implantable device 102 deploys from the second intermediate point (C) toward the proximal portion (D) so as to deploy the implantable device 102 from the partially deployed configuration to the expanded, deployed configuration.

In some examples, the first connected portion 110 of the secondary constraining sleeve 108 is connected first, from the first intermediate point (B) to the distal portion (A) using a first deployment line 400. A second deployment line 402 is connected from the second intermediate point (C) to the proximal portion (D) and then woven through the seamlines and the middle unused stitch holes of the secondary constraining sleeve 108. This configuration results in simultaneous distal-to-middle deployment and proximal-to-middle deployment, with the opposing deployment directions on the proximal and distal portions of the secondary constraining sleeve 108 causing the end portions of the implantable device 102 to deploy inwardly towards the midsection of the device 102. This configuration simplifies the manufacturing process for the sleeve as compared to the embodiment shown in FIG. 3 while still achieving the same deployment results and having the inner-crushed device. As the implantable device 102 deploys from the distal portion (A) and the proximal portion (D), the device 102 assumes a greater diameter than the midsection (B and D) and thereby pushes (or, alternatively, “shrugs” or “splits”) the secondary constraining sleeve 108 away from the proximal and distal portions (A and D).

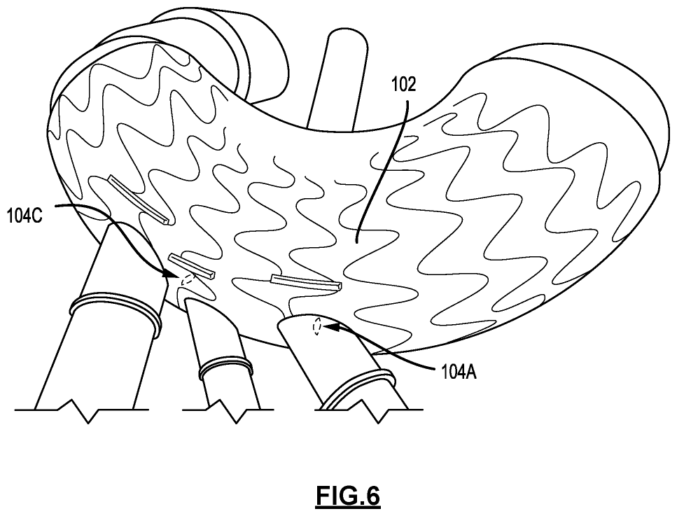

FIG. 5 shows the implantable device 102 when partially deployed in the intermediate deployment configuration, or a partially deployed configuration. FIG. 6 shows the implantable device 102 when fully deployed in the expanded, deployed configuration, or a fully deployed configuration. The BCA opening 104A, the LSA opening 104B, and the LCCA opening 104C are visible in FIG. 5, as are the opposing edges 116, 118 of the secondary constraining sleeve 108. In FIG. 6, the BCA opening 104A and the LCCA opening 104C are visible.

Processes of manufacturing the endoluminal device are also disclosed herein. Referring to FIG. 8, a manufacturing process 800 may be a method that includes a step 802 of providing an implantable device with at least one opening providing access to an inner lumen of the implantable device. For example, the implantable device may have a first end portion, a second end portion, and an intermediate portion located between the first and second end portions, and the intermediate portion may have the opening(s) providing access to the inner lumen of the implantable device. The implantable device may be transitionable between a collapsed, delivery configuration and an expanded, deployed configuration. The method also includes a step 804 of disposing a material around the implantable device, and a step 806 of partially connecting together opposing edges of the material to form a first connected portion including a distal portion of the sleeve, a second connected portion including a proximal portion of the sleeve, and an unconnected portion located between the first and second connected portions providing access to the opening(s) in the collapsed, delivery configuration. The material may be a single sheet, a single membrane, multiple sheets, multiple membranes, or any suitable combination thereof. The connecting of the opposing edges in step 806 can be achieved in any suitable method, as explained herein.

In some examples, step 806 includes a step of releasably coupling together the opposing edges of the sleeve using a single line to constrain the implantable device in the collapsed, delivery configuration. In some examples, the step includes forming, using the single line, the first connected portion of the sleeve by connecting together the opposing edges from the distal portion to the first intermediate point, and forming, using the single line, the second connected portion by connecting together the opposing edges from the second intermediate point to the proximal portion. In some examples, the step includes forming, using the single line, the first connected portion of the sleeve by connecting together the opposing edges from the first intermediate point to the distal portion, and forming, using the single line, the second connected portion by connecting together the opposing edges from the second intermediate point to the proximal portion. The first intermediate point is located between the distal portion and the proximal portion, and the second intermediate point is located between the distal portion and the proximal portion.

In some examples, step 806 includes a step of releasably coupling together the opposing edges of the sleeve using a first line and a second line to constrain the implantable device in the collapsed, delivery configuration. In some examples, the step includes forming, using the first line, the first connected portion of the sleeve by connecting together the opposing edges from the first intermediate point to the distal portion, and forming, using the second line, the second connected portion by connecting together the opposing edges from the second intermediate point to the proximal portion. The first and second lines may be separately retractable. In some examples, the opening(s) may include at least a first opening, a second opening, and a third opening.

In some examples, the manufacturing process includes a step of stitching together (using a suitable tool such as a chain stitch) two sides of a longitudinal edge of a secondary sleeve, starting at about 60 mm from the proximal edge of the sleeve and stitching toward the proximal sleeve edge. The process includes a step of tying two slip knots at the proximal sleeve edge so as to leave a loop of the second slip knot long, after which this loop is secured, for example using a lock clip. In a subsequent step, the deployment line is weaved through the single stitches of the connected sleeve, starting from the secured slip knots, so as to place the weave at about 2 mm in length along the longitudinal length of the sleeve. In a subsequent step, the weaving is continued on a single stitch sleeve side only, capturing unused stitch holes at about every 2 mm for a total length of about 64 mm along the longitudinal length of the sleeve. In a subsequent step, the two sides of the longitudinal edge of the secondary sleeve are stitched together (using the chain stitch, for example) starting from the end of the woven sleeve section and stitching toward the distal sleeve edge. At least about 10 cm of the sleeve is to be stitched this way. In a subsequently step, two slip knots are tied, leaving a loop of second slip knot long, after which this loop is tucked under the fourth double stitch from the distal edge of the sleeve, and the loop is secured (using a lock clip, for example). Lastly, a step includes winding the remaining portion of the deployment line onto a bobbin for storage.

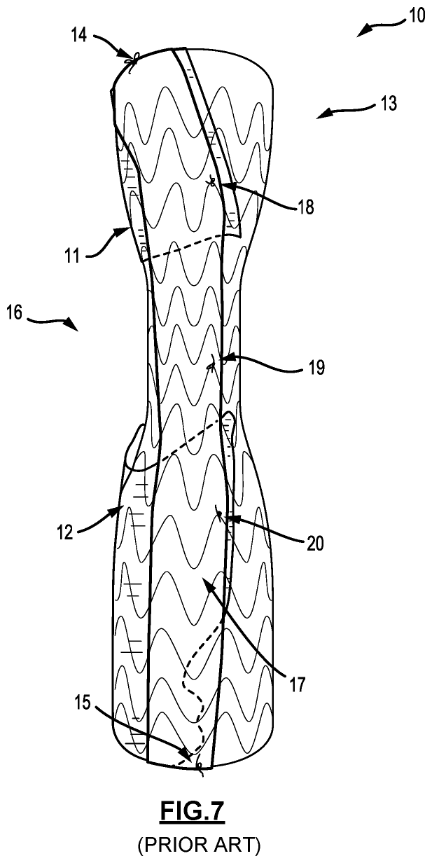

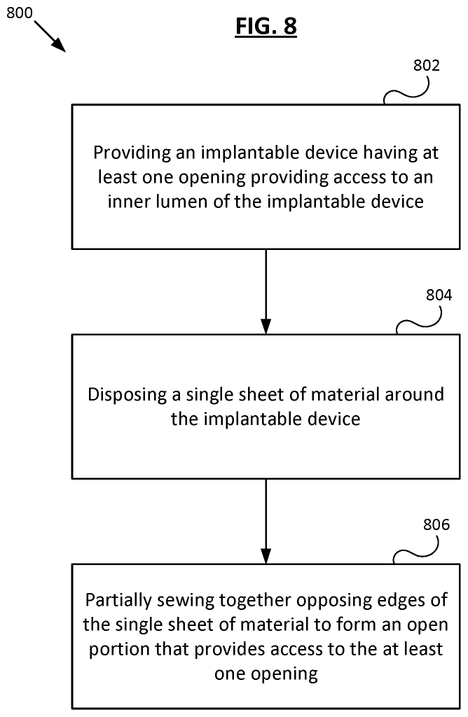

Currently, prior-art endoluminal devices as known in the art require two or more separate sleeves that separately facilitate the crushing of an implantable device. For example, as shown in FIG. 7, a prior-art endoluminal device 10 is provided with a proximal sleeve 11 and a distal sleeve 12 such that one end portion (proximal end portion 14) of the implantable device 13 is constrained using the proximal sleeve 11, and the other end portion (distal end portion 15) of the implantable device 13 is constrained using the distal sleeve 12, leaving a middle portion 16 that is not entirely covered by either of the two (or more, in some examples) sleeves 11 and 12 to provide access to any opening (not shown) that are formed in the implantable device 13 that provide access to its inner lumen within the implantable device 13. Also shown is an outer sleeve 17 positioned over the proximal sleeve 11 and the distal sleeve 12. Attachments 18, 19, and 20 are also provided to attach the sleeves to the implantable device 13. Attachments are also be provided at the end portions 14 and 15.

However, using two or more separate sleeves to facilitate the crushing of the implantable device introduces uncertainties to the endoluminal device that are not immediately noticeable due to the location of the proximal sleeve being in a region of the device that is different from that of the predicate devices. One such observed issue is that a line or a tether that is used for deployment of the device can be inadvertently captured at an un-taped apex tip of one of the stent components during deployment. Another issue is that, if an apex portion of one of the stent components is bent backward and is not covered with any sleeve, the backwardly-bent portion can be caught in a funnel during or after deployment. Addressing such uncertainties as they are learned leads to increased manufacturing complexity.

Furthermore, in prior-art devices, the customization process of two or more sleeves and then routing the deployment lines would increase the time required to do so, as compared to the predicate devices with similar processes such as CMDS and ASG, both of which are available from or manufactured by W. L. Gore & Associates, Inc. In some cases, the customization of two or more sleeves and the routing of two or more deployment lines also increase the ergonomic impact for these steps as compared to predicate devices with similar processes. In some cases, the customization, the crushing, and the routing (or fiber management) of two or more deployment lines introduces additional amount of potential impacts to yield, thereby allowing errors to occur during the manufacturing process. In some cases, the use of two or more sleeves increases the material cost and material waste associated with the sleeves as compared to the predicate products with similar processes. In some cases, un-taped apices, or the apices of the stent components that are not covered by the sleeves, may present additional risks, such as the edge of the sleeves being caught by a tip of the un-taped apex.

Risks of such problems as listed above can be minimized or reduced using the secondary constraining sleeve 108 with the unconnected portion 114 for constraining the implantable device 102 as disclosed herein. For example, the risk of crushing the implantable device 102 into two or more sleeves with yet unknown uncertainties (e.g., uncertainties related to the sleeves' seamlines) can be addressed by aligning the unconnected portion 114 with the opening(s) 104. In manufacturing, the takt time (that is, the required product assembly duration that needed to match the demand for the product) of the endoluminal device 100 can be reduced to be comparable to the predicate devices (such as CMDS and ASG). Also, the ergonomic impacts of sleeve customization can remain consistent with the aforementioned predicate devices. The anticipated yield impacts can also remain consistent with the aforementioned predicate devices. The amount of material cost and material waste can also remain consistent with the aforementioned predicate devices. Furthermore, using the sleeve 108 to cover the un-taped apex region of the implantable device 102 (that is, the apices of the stent components 120) allows multibranch arch (“MBA”) graft devices to be aligned with the aforementioned predicate devices, thereby allowing for alignment on future improvements. Additionally, doing so removes the risk of an edge of any additional outer sleeve to be caught on an un-taped apex tip of the stent components 120 while the additional outer sleeve moves away from the opening region during deployment. Such risk is not only minimized for the MBA devices using the embodiments disclosed herein, but also for future improvements to the attachment locations for the additional outer sleeve. In some examples, the lengths of the first connected portion 110, the second connected portion 112, and the unconnected portion 114 may correspond with the proximal section, the distal portion, and the opening portion of the MBA device, and, with regards to the openings 104, the BCA opening, the LSA opening, and the LCCA opening may correspond to the openings of the MBA device.

In addition, the prior-art endoluminal devices still require customization of the sleeve on the backend manufacturing line which may increase the likelihood for error and increase the takt time. In comparison, the endoluminal device as disclosed herein only requires a secondary constraining sleeve, which reduces the material usage as compared to the prior-art devices. Also, the device as disclosed herein keeps the un-taped midsection apices of the implantable device covered with the secondary constraining sleeve through outer crush process, thereby addressing the uncertainties that may be observed in the backend manufacturing with the prior-art devices. The device as disclosed herein addresses some uncertainties observed with the outer sleeve retraction, allowing for further understanding of other manufacturability solutions.

In some examples, a method of operating an endoluminal device 100 may include a step of delivering the endoluminal device such that an implantable device is disposed in a collapsed, delivery configuration or a partially deployed configuration at a location within a vasculature. The implantable device has at least one opening providing access to an inner lumen of the implantable device. The implantable device has a secondary constraining sleeve disposed around the implantable device. The sleeve has a first connected portion including a distal portion of the secondary constraining sleeve, a second connected portion including a proximal portion of the secondary constraining sleeve, and an unconnected portion located between the first and second connected portions. The unconnected portion is adjacent to or overlaying the at least one opening when in the partially deployed configuration operable to provide access to the at least one opening from outside of the secondary constraining sleeve. The method may also include a step of at least partially removing the secondary constraining sleeve from the implantable device to cause the implantable device to transition from the partially deployed configuration to an expanded, deployed configuration at the location within the vasculature.

Furthermore, the endoluminal device as disclosed herein facilitates simple and precise delivery of a multi-branch endograft through staged deployment, as well as facilitating passive rotational alignment and femoral branch delivery, and also providing reliable opening access through rail guides. The device also provides durable arch treatment and has broad clinical applicability with retrograde openings that facilitate broad anatomical fit, such as for thoracic aneurysm, chronic dissections, anastomotic pseudoaneurysm after previous surgical repair of the thoracic aorta or aortic heart valve, and penetrating atherosclerotic aortic ulcer (PAU) with or without aortic intramural hematoma (IMH). The device may be implemented with other medical treatment products and devices such as ASG, MBA devices, TBE, VBX endoprosthesis, and CTAG. The device facilitates minimizing or reducing the risk of stroke by reducing manipulation via passive rotational alignment and simple opening access, providing retrograde branch delivery, and providing compatibility with embolic protection system (EPS).

It is further understood that sleeves such as the secondary constraining sleeve may be implemented not only as a secondary or second sleeve, but may be implemented as the only sleeve constraining a device or may be used with additional sleeves beyond what is described herein.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An endoluminal device comprising:
an implantable device transitionable between a collapsed, delivery configuration, an expanded, deployed configuration, and a partially deployed configuration between the collapsed, delivery configuration and the expanded, deployed configuration, the implantable device having at least one opening providing access to an inner lumen of the implantable device; and
a constraining sleeve removably disposed around the implantable device, the constraining sleeve having a first connected portion including a distal portion of the constraining sleeve, a second connected portion including a proximal portion of the constraining sleeve, and an unconnected portion located between the first and second connected portions, the unconnected portion being adjacent to or overlaying the at least one opening when in the partially deployed configuration operable to provide access to the at least one opening from outside of the constraining sleeve.

2. The endoluminal device of claim 1, wherein the implantable device is self-expanding.

3. The endoluminal device of claim 1, wherein the constraining sleeve has two opposing edges, a first intermediate point located between the distal portion and the proximal portion, and a second intermediate point located between the distal portion and the proximal portion, and wherein the constraining sleeve has the first connected portion between the distal portion and the first intermediate point of the constraining sleeve, and the second connected portion between the proximal portion and the second intermediate point of the constraining sleeve.

4. The endoluminal device of claim 3, further comprising:
a single line releasably coupling together the two opposing edges of the constraining sleeve to constrain the implantable device in the partially deployed configuration.

5. The endoluminal device of claim 4, wherein the line forms the first connected portion of the constraining sleeve by connecting together the two opposing edges from the distal portion to the first intermediate point, the line forming the second connected portion by connecting together the two opposing edges from the second intermediate point to the proximal portion.

6. The endoluminal device of claim 5, wherein proximally retracting the line causes the implantable device to deploy from the distal portion toward the first intermediate point and to deploy from the second intermediate point toward the proximal portion.

7. The endoluminal device of claim 4, wherein the line connects together the two opposing edges from the first intermediate point to the distal portion defining the first connected portion of the constraining sleeve, and the line connects together the two opposing edges from the second intermediate point to the proximal portion defining the second connected portion.

8. The endoluminal device of claim 7, wherein proximally retracting the line causes the line to become unconnected or disengaged from the two opposing edges such that the implantable device deploys from the first intermediate point toward the distal portion and deploys from the second intermediate point toward the proximal portion so as to deploy the implantable device from the partially deployed configuration to the expanded, deployed configuration.

9. The endoluminal device of claim 3, further comprising:
a first line and a second line releasably coupling together the two opposing edges of the first and second connected portions, respectively, to constrain the implantable device in the partially deployed configuration, the first line connecting together the two opposing edges from the first intermediate point to the distal portion defining the first connected portion of the constraining sleeve, and the second line connecting together the two opposing edges from the second intermediate point to the proximal portion defining the second connected portion.

10. The endoluminal device of claim 9, wherein the first and second lines are separately retractable.

11. The endoluminal device of claim 9, wherein proximally retracting the first line causes the first line to become unconnected or disengaged from the two opposing edges such that the implantable device deploys from the first intermediate point toward the distal portion and proximally retracting the second line causes the second line to become unconnected or disengaged from the two opposing edges such that the implantable device deploys from the second intermediate point toward the proximal portion so as to deploy the implantable device from the partially deployed configuration to the expanded, deployed configuration.

12. The endoluminal device of claim 1, wherein the at least one opening includes at least a first opening, a second opening, and a third opening.

13. A method of manufacturing an endoluminal device, the method comprising:
providing an implantable device transitionable between a collapsed, delivery configuration, an expanded, deployed configuration, and a partially deployed configuration between the collapsed, delivery configuration and the expanded, deployed configuration, the implantable device having at least one opening providing access to an inner lumen of the implantable device;
disposing a material around the implantable device; and
partially connecting together opposing edges of the material to form a first connected portion including a distal portion of the constraining sleeve, a second connected portion including a proximal portion of the constraining sleeve, and an unconnected portion located between the first and second connected portions providing access to the at least one opening in the partially deployed configuration.

14. The method of claim 13, the partially connecting together opposing edges of the material comprises:
releasably coupling together the opposing edges of the constraining sleeve using a single line to constrain the implantable device in the partially deployed configuration.

15. The method of claim 14, the partially connecting together opposing edges of the material comprises:
forming, using the single line, the first connected portion of the constraining sleeve by connecting together the opposing edges from the distal portion to a first intermediate point; and
forming, using the single line, the second connected portion by connecting together the opposing edges from a second intermediate point to the proximal portion,
wherein the first intermediate point is located between the distal portion and the proximal portion, and the second intermediate point is located between the distal portion and the proximal portion.

16. The method of claim 14, the partially connecting together opposing edges of the material comprises:
forming, using the single line, the first connected portion of the constraining sleeve by connecting together the opposing edges from a first intermediate point to the distal portion; and
forming, using the single line, the second connected portion by connecting together the opposing edges from a second intermediate point to the proximal portion,
wherein the first intermediate point is located between the distal portion and the proximal portion, and the second intermediate point is located between the distal portion and the proximal portion.

17. The method of claim 13, the partially connecting together opposing edges of the material comprises:
releasably coupling together the opposing edges of the constraining sleeve using a first line and a second line to constrain the implantable device in the partially deployed configuration.

18. The method of claim 17, the partially connecting together opposing edges of the material comprises:
forming, using the first line, the first connected portion of the constraining sleeve by connecting together the opposing edges from a first intermediate point to the distal portion; and
forming, using the second line, the second connected portion by connecting together the opposing edges from a second intermediate point to the proximal portion,
wherein the first intermediate point is located between the distal portion and the proximal portion, and the second intermediate point is located between the distal portion and the proximal portion.

19. The method of claim 18, wherein the first and second lines are separately retractable.

20. The method of claim 13, wherein the at least one opening includes at least a first opening, a second opening, and a third opening.

21. A method of operating an endoluminal device, the method comprising:
delivering the endoluminal device such that an implantable device is disposed in a collapsed, delivery configuration or a partially deployed configuration at a location within a vasculature, the implantable device having at least one opening providing access to an inner lumen of the implantable device, the implantable device having a constraining sleeve disposed around the implantable device, the constraining sleeve having a first connected portion including a distal portion of the constraining sleeve, a second connected portion including a proximal portion of the constraining sleeve, and an unconnected portion located between the first and second connected portions, the unconnected portion being adjacent to or overlaying the at least one opening when in the partially deployed configuration operable to provide access to the at least one opening from outside of the constraining sleeve; and
at least partially removing the constraining sleeve from the implantable device to cause the implantable device to transition from the partially deployed configuration to an expanded, deployed configuration at the location within the vasculature.
